(19) United States
(12) Patent Application Publication
(10) Pub. No.: US 2005/0010231 A1
(43) Pub. Date: Jan. 13, 2005

(54) Method and apparatus for strengthening the biomechanical properties of implants

(76) Inventor: Thomas H. Myers, Marietta, GA (US)

Correspondence Address: ALSTON & BIRD LLP;BANK OF AMERICA PLAZA, 101 SOUTH TRYON STREET, SUITE 4000, CHARLOTTE, NC, 28280-4000, US

(21) Appl. No.: 10/873,537

(22) Filed: Jun. 21, 2004

Related U.S. Application Data
(60) Provisional application No. 60/479,850, filed on Jun. 20, 2003.

(51) Int. Cl. A61B 17/58

(52) U.S. Cl. 606/86

(57) ABSTRACT

A method for agitating a surgical fluid using a vibrating probe is disclosed. The agitation method drives entrapped air voids out of the surgical fluid and forces the fluid into a plurality of pores of various sizes in the adjacent bone. The vibrating apparatus in one embodiment includes a probe tip disposed upon a graspable elongate shaft and a series of fins extending into the fluid. The apparatus in one embodiment may include a set of probe tips of different shapes and sizes. The agitation and interdigitation method may facilitate any procedure involving any type of surgical fluid, with or without a prosthetic device such as an intramedullary nail or femoral prosthesis. This Abstract is provided to quickly inform a reader about the subject matter, and not for use interpreting the scope or meaning of the claims.

### CROSS-REFERENCE TO RELATED APPLICATIONS

This application claims the benefit and priority of a U.S. provisional application for patent entitled, “Method and Apparatus for Improving Bond Strength Between Prosthetic and Bone,” filed Jun. 20, 2003, and assigned Application No. 60/479,850, which is incorporated herein by reference in its entirety.

### BACKGROUND

1. Technical Field

The following disclosure relates generally to the field of orthopaedic medicine and, more particularly, to a method and apparatus for removing entrapped air voids from a surgical fluid such as bone cement and otherwise improving the bond strength and durability between a surgical fluid and natural bone, and between a surgical fluid and a prosthesis.

2. Description of Related Art

Many surgical techniques for treating a bone defect use a surgical fluid. A bone defect may be caused by a fracture or other bone insult caused by trauma, a void or other defect caused by infection, tumor, a degenerative process, or another bone pathology. Among the various surgical fluids in use today are polymer bone cements, viscous bone grafts, natural or synthetic bone graft substitutes, de-mineralized bone matrix, collagen-based matrices, and solidifying gels and putties.

When a surgical fluid is used for the repair and treatment of a bone defect, the integrity of the fluid after hardening and the strength of its bond to the adjacent bone mass are critical factors in providing a stable and durable treatment. The surface where a surgical fluid meets the bone mass may be referred to as the fluid-bone interface. Similarly, the surface where a surgical fluid meets a prosthesis may be referred to as the fluid-prosthesis interface. When subjected to the ordinary physiological loads of daily activity, a prosthesis or solidified fluid mass must be capable of successfully transferring the load to the bone without causing a fracture or loosening at the interface. A failure or weakening of the bond at the interface will inevitably cause complications requiring additional surgical correction.

The integrity of a mass or column of surgical fluid may be hindered by the presence of air voids in the fluid. Currently, air voids are removed from surgical fluid by centrifugation or vacuum mixing before the fluid is placed into an injecting gun and inserted into a bone canal or cavity. Such fluid preparation techniques, however, do not remove air voids that may be introduced when the fluid is injected into a bone canal or other cavity. For this and other reasons, questions remain about the benefit of such fluid preparation techniques performed in the operating room before injection.

The use of bone cement as a surgical fluid has evolved in surgical procedures. First-generation cement application involved the technique of finger-packing a doughy cement into an unplugged bony cavity, such as the femoral canal. Long-term results reported a thirty to forty percent aseptic loosening rates in studies having an eight- to ten-year duration. Second-generation techniques included plugging the femoral canal, cleaning the canal with pulsed lavage, and following with a retrograde injection of bone cement with a cement gun.

Third-generation techniques involve all aspects of the second-generation approach, plus the reduction of entrapped air in the fluid using vacuum mixing and centrifugation. Pressurization of the cement mantle and surface modification of the femoral components by such methods as grit blasting and PMMA pre-coating were performed to enhance the bond at the prosthesis-fluid interface. In studies, the ultimate tensile strength of the cement column was increased twenty-four percent, while compressive strength increased one hundred, thirty-six percent. The reduction or removal of air voids takes on additional importance after an analysis of failed femoral components revealed the cement fractures occurred through the air voids. In vivo studies of bone cement prepared using centrifugation before injection have shown a reduction in air void size from four hundred microns to two hundred microns. However, as mentioned above, large pores and air voids removed during centrifugation may be re-introduced into the cement column during injection into the bone canal. Moreover, the technique of pressurizing the cement mantel does not alleviate the presence of air voids in vivo.

Fourth-generation techniques include all aspects of the third-generation approach, plus the addition of proximal and distal femoral cement centralizers on the prosthesis. In use, a centralizer may be fitted and installed at the distal end of a bone canal, where a centrally-disposed recess may be sized and shaped to receive the distal end of a prosthesis. Although such a post-in-hole centralizer may provide improved alignment, the technical challenges of bond strength and air void reduction remain to be solved.

Thus, there exists a need in the art for a method of eliminating or dispersing air voids from a surgical fluid in vivo; in other words, after the cement or other fluid has been injected into a bone canal or other cavity. There is also a need in the art to improve bond strength between surgical fluids and the surrounding bone, and between surgical fluids and any prosthetic device in use.

Certain illustrative and exemplary apparatuses, systems, and methods are described herein in connection with the following description and the accompanying drawing figures. The examples discussed represent only a few of the various ways of applying the principles supporting the material disclosed and, thus, the examples are intended to include equivalents. Other advantages and novel features may become apparent from the detailed description which follows, when considered in conjunction with the drawing figures.

### SUMMARY OF THE INVENTION

The following summary is not an extensive overview and is not intended to identify key or critical elements of the apparatuses, methods, systems, processes, and the like, nor is it intended to delineate the scope of such elements. This Summary provides a conceptual introduction in a simplified form as a prelude to the more-detailed description that follows.

The example methods, products, and systems described herein may improve the structural integrity of a column or mass of surgical fluid adjacent a bony surface.

In one aspect, the present invention may include a method of improving the structural integrity of a column of surgical fluid adjacent a bone wall having a plurality of open pores, including the steps of inserting a probe into the column of surgical fluid and vibrating the probe within the column of surgical fluid in order to drive entrapped air toward and through a surface of the fluid and to drive the surgical fluid into one or more of the plurality of open pores.

The method may also include the steps of inserting a restrictor to form a base for the column of surgical fluid, capping the column of surgical fluid near the surface, and drawing a partial vacuum above the colunm of surgical fluid in order to help draw the entrapped air toward and through the surface. The method may also include touching the probe to the restrictor in order to induce vibrations within the restrictor and within the bone wall. The method may also include touching the probe to a bone wall in order to induce vibrations within the bone wall.

The method may also include manipulating the probe from near the base of the column of surgical fluid toward the surface. The method may also include manipulating the probe near the bone wall in order to improve the depth of interdigitation between the fluid and the plurality of open pores.

The method may also include cleansing the bone wall and then drying the bone wall, before injecting the column of surgical fluid. The method may also include varying the frequency of vibration of the probe or varying the amplitude of vibration of the probe.

In another aspect, the present invention may include a method of removing entrapped air from a column of surgical fluid, including the steps of inserting a probe into the column of surgical fluid and vibrating the probe within the column of surgical fluid in order to drive entrapped air toward and through a surface of the fluid. The method may also include inserting a restrictor to form a base for the column of surgical fluid, capping the column of surgical fluid near the surface, and drawing a partial vacuum above the column of surgical fluid in order to help draw the entrapped air toward and through the surface.

The method may also include manipulating the probe from near the base of the column of surgical fluid toward the surface. The method may also include varying the frequency of vibration of the probe or varying the amplitude of vibration of the probe.

In another aspect, the present invention may include a method of provoking interdigitation between a column of surgical fluid and a bone wall having a plurality of open pores, wherein the method includes the steps of inserting a probe into the column of surgical fluid and vibrating the probe within the column of surgical fluid in order to drive the surgical fluid into one or more of the plurality of open pores.

The method may also include inserting a restrictor to form a base for the column of surgical fluid, capping the column of surgical fluid near the surface, and drawing a partial vacuum above the column of surgical fluid in order to help draw the entrapped air toward and through the surface.

The method may also include touching the probe to the restrictor in order to induce vibrations within the restrictor and within the bone wall. The method may also include touching the probe to the bone wall in order to induce vibrations within the bone wall.

The method may also include cleansing the bone wall and then drying the bone wall, before injecting the column of surgical fluid.

The method may also include manipulating the probe near the bone wall in order to improve the depth of interdigitation between the fluid and the plurality of open pores. The method may also include varying the frequency of vibration of the probe or varying the amplitude of vibration of the probe.

In another aspect, the present invention may include a vibrating probe apparatus for agitating a surgical fluid, comprising an elongate shaft having a proximal end and an opposing distal end, the proximal end being graspable for supporting and maneuvering the apparatus; a probe tip disposed upon the shaft near the distal end; and a motor for producing a vibration within the probe tip. Also, the motor may produce a vibration along the elongate shaft.

The apparatus may also include one or more fins disposed about the probe tip and extending into the fluid. The apparatus may also include one or more fins disposed about the elongate shaft and extending into the fluid.

The apparatus may also include a graspable handle disposed upon the shaft near the proximal end for supporting and maneuvering the apparatus.

The apparatus may also include a cable for coupling the motor to the apparatus.

The apparatus may also include a controller coupled to the motor and configured to vary the frequency or amplitude of the vibration.

In another aspect, the present invention may include a system for bonding a column of surgical fluid to a bone wall, the system comprising a fluid restrictor positioned to form a base for the column of surgical fluid; a plurality of open pores along the bone wall; and a zone of interdigitation between the surgical fluid and the plurality of open pores, the zone promoted by the temporary insertion of a vibrating probe configured to drive the surgical fluid into one or more of the plurality of open pores, the vibrating probe powered by a motor and comprising an elongate shaft having a graspable proximal end and an opposing distal end, a probe tip disposed upon the shaft near the distal end.

The system may also include a partial vacuum zone adjacent an outer surface of the column of surgical fluid for drawing one or more air voids within the surgical fluid toward the outer surface, the partial vacuum zone created by a suction device disposed through a cap at least partially sealing the column of surgical fluid near the outer surface. Also, the vibrating probe may be used to mobilize the one or more air voids toward the outer surface. The system may also include a lug disposed upon the fluid restrictor and configured to be releasably attached to the distal end of the elongate shaft.

In another aspect, the present invention may include a system for consolidating (1) a prosthesis, (2) a column of surgical fluid, and (3) a porous bone wall into an integrated structure, the system comprising a fluid restrictor positioned to form a base for the column of surgical fluid; a plurality of open pores along the bone wall; a first zone of interdigitation between the surgical fluid and the plurality of open pores, the first zone promoted by the temporary insertion of a vibrating probe configured to drive the surgical fluid into one or more of the plurality of open pores; and a second zone of interdigitation between the outer surface of the prosthesis and the surgical fluid, the second zone promoted by the temporary insertion of a vibrating probe configured to drive the surgical fluid into the outer surface; the vibrating probe powered by a motor and comprising an elongate shaft having a graspable proximal end and an opposing distal end, a probe tip disposed upon the shaft near the distal end.

The system may also include a partial vacuum zone adjacent an outer surface of the column of surgical fluid for drawing one or more air voids within the surgical fluid toward the outer surface, the partial vacuum zone created by a suction device disposed through a cap at least partially sealing the column of surgical fluid near the outer surface. Also, the vibrating probe may be used to mobilize the one or more air voids toward the outer surface. The system may also include a lug disposed upon the fluid restrictor and configured to be releasably attached to the distal end of the elongate shaft.

These and other objects are accomplished by the methods, products, and systems described herein and will become apparent from the following description of a preferred embodiment in conjunction with the accompanying drawings in which like numerals designate like elements.

### BRIEF DESCRIPTION OF THE DRAWING

The invention may be more readily understood by reference to the following description, taken with the accompanying drawing figures, in which:

FIG. 1 is an illustration of an apparatus in use within a long bone, according to one embodiment of the present invention.

FIG. 2 is a cross-sectional illustration of an apparatus within an intramedullary canal, according to one embodiment of the present invention.

FIG. 3 is a cross-sectional illustration of a prosthetic within an intramedullary canal, according to one embodiment of the present invention.

FIG. 4 is an illustration of an apparatus in use within a long bone, according to one embodiment of the present invention.

FIG. 5 is an illustration of a probe tip and fins for an apparatus, according to one embodiment of the present invention.

FIG. 6 is an illustration of a probe tip and fins for an apparatus, according to one embodiment of the present invention.

FIG. 7 is an illustration of a probe tip and fins for an apparatus, according to one embodiment of the present invention.

### DETAILED DESCRIPTION

This application claims the benefit and priority of a U.S. provisional application for patent entitled, “Method and Apparatus for Improving Bond Strength Between Prosthetic and Bone,” filed Jun. 20, 2003, and assigned Application No. 60/479,850, which is incorporated herein by reference in its entirety.

1. Introduction

Exemplary systems, methods, and apparatuses are now described with reference to the drawing figures, where like reference numerals are used to refer to like elements throughout the several views. In the following description, for purposes of explanation, numerous specific details are set forth in order to facilitate a thorough understanding of the systems, methods, apparatuses, and the like. It may be evident, however, that the exemplars described may be practiced without these specific details. In other instances, common structures and devices are shown in block diagram form in order to simplify the description. Indeed, this invention may be embodied in many different forms and should not be construed as limited to the embodiments set forth herein; rather, these embodiments are provided so that this disclosure will satisfy applicable legal requirements.

Many modifications and other embodiments may come to mind to one skilled in the art who has the benefit of the teachings presented in the description and drawings. It should be understood, therefore, that the invention is not be limited to the specific embodiments disclosed and that modifications and alternative embodiments are intended to be included within the scope of the disclosure and the exemplary inventive concepts. Although specific terms may be used herein, they are used in a generic and descriptive sense only and not for purposes of limitation.

2. Definitions

Surgical fluids are used in a wide variety of applications. As used herein, the term “surgical fluid” may include polymer bone cement, viscous bone grafts, natural or synthetic bone graft substitutes, de-mineralized bone matrix, collagen-based matrices, solidifying gels and putties, and other substances having a low viscosity that are used in procedures where they are applied to bone for such purposes as bonding, stabilization, fixation, as a bone substitute, or for other therapeutic purposes. The term surgical fluid is also intended to include and encompass those fluids yet to be invented or discovered which have a curative, medicinal, or therapeutic use inside the body.

A surgical fluid is a type of implant. The noun “implant,” as used herein, is intended to refer to something implanted, especially in body tissue, such as a column or mass of surgical fluid, a bone graft, a stabilizing rod or internal support, or a prosthetic joint or bone segment.

As used herein, the verb “consolidate” and its related forms are intended to refer to the joining together of two or more items into one unit or into a coherent whole, to make firm or more secure, and to strengthen.

As used herein, the term “interdigitation” is intended to refer to the act of interlocking or the condition of being interlocked or interpenetrated.

As used herein, the noun “probe” is intended to refer to an instrument that generally consists of an elongate, generally slender shaft that is typically flexible and pointed to facilitate insertion and exploration within a passage or cavity.

Furthermore, to the extent that the term “includes” is employed in the detailed description or the claims, it is intended to be inclusive in a manner similar to the term “comprising” as that term is interpreted when employed as a transitional word in a claim. Further still, to the extent that the term “or” is employed in the claims (for example, A or B) it is intended to mean “A or B or both.” When the author intends to indicate “only A or B but not both,” the author will employ the phrase “A or B but not both.” Thus, use of the term “or” herein is the inclusive use, not the exclusive use. See Bryan A. Garner, A Dictionary Of Modern Legal Usage 624 (2d ed. 1995).

3. Bone Physiology and Treatment Techniques

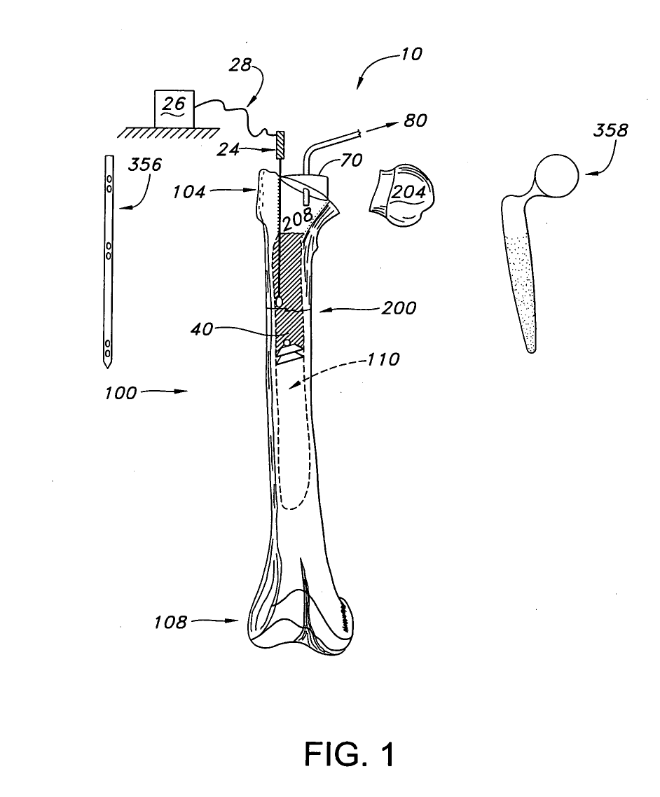

The bone 100 illustrated in FIG. 1 has a proximal end 104, a distal end 108, and an intramedullary canal 110. A bone insult 200 is depicted as a fracture line, but it may be a void or another type of bone defect caused, for example, by trauma, infection, a tumor, or a degenerative process. A femur is illustrated in FIG. 1, and the femoral head has been removed or resected. Several surgical techniques result in a resected bone end 204, as shown. Although the bone in FIG. 1 is a femur, the present invention may be used for procedures involving any bony or porous structure to improve the integrity and strength of a mass of surgical fluid 40 adjacent the structure.

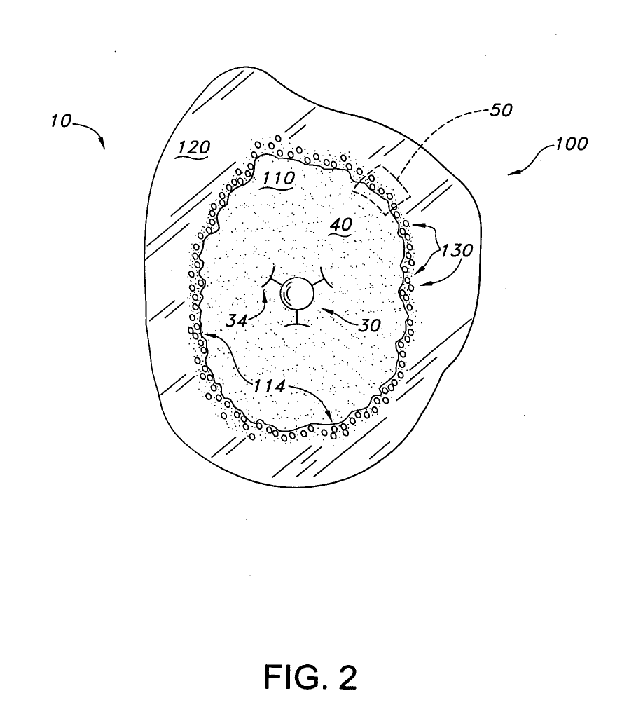

FIG. 2 is a cross-sectional illustration of a bone 100, showing various bony features including the bone wall 120, the endosteum 114 or inner lining of the intramedullary canal 110, and a plurality of pores 130 of various shapes and sizes.

Referring again to FIG. 1, many surgical techniques for treating a bone defect or insult 200 may use a surgical fluid 40. Among the various surgical fluids in use today are polymer bone cements such as PMMA (polymethylmethacrylate), viscous bone grafts, natural or synthetic bone graft substitutes, de-mineralized bone matrix, collagen-based matrices, and solidifying gels and putties. The surgical fluid 40 may injected into the intramedullary canal 110 or other void or space using, for example, a cement gun (not shown). In order to gain access to an interior bony space, the surgeon may created a reamed canal 208 through which instruments and/or prosthetic devices may be inserted. FIG. 1 shows a reamed canal 208 through the bone at the location where a resected bone end 204 has been removed. The reamed canal 208 may be drilled at an alternate location, directly through the bone wall for example, depending upon the desired route of access into the canal 110.

Implantation of a femoral prosthesis is one surgical procedure where the apparatus and method of the present invention may be applied. One type of femoral prosthesis 358 is illustrated in FIG. 1.

Fracture fixation is another surgical procedure where the apparatus and method of the present invention may be applied. Another type of prosthesis 350 called an intramedullary nail 356 is illustrated in FIG. 1. In use, the nail prosthesis 356 may be placed along the length of a fractured bone in order to maintain proper alignment. The nail 356 may be secured by screws and will act as a load-bearing device until the fracture or other bone insult 200 is healed.

Other surgical procedures, including those with or without various prosthetic devices, may benefit from the advantages of the apparatus and method of the present invention.

4. A Vibrating Probe

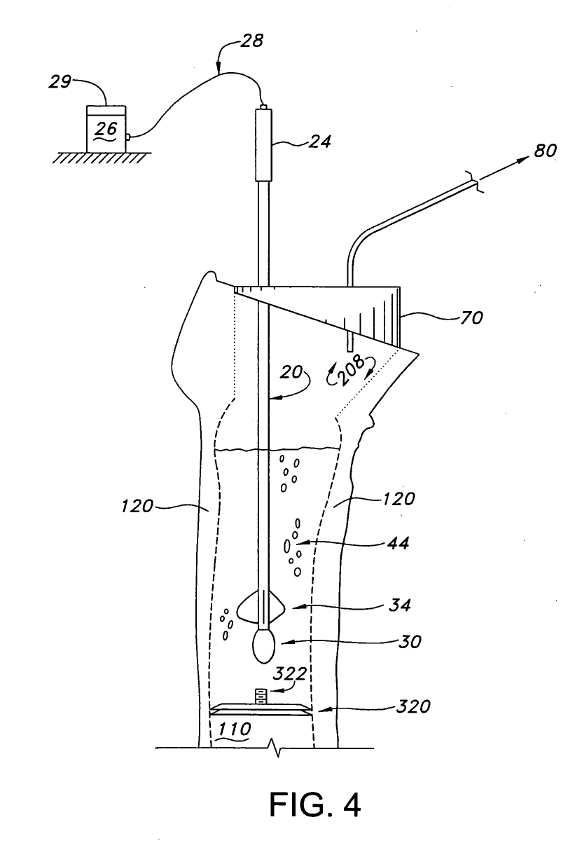

FIG. 4 is an illustration of an apparatus 10 according to one embodiment of the present invention. The apparatus 10 may include an elongate shaft 20 with, in one embodiment, a vibrating probe tip 30 on one end. In one embodiment, the shaft 20 may be generally rigid and may include a graspable handle 24. Also, the shaft 20 itself may be graspable along its length. The apparatus 10 may include a motor 26 coupled to the shaft 20 by a cable 28. For some applications, the motor 26 may be built into the probe tip 30 itself.

In one embodiment, the apparatus 10 may include an elongate shaft 20, with a probe tip 30 on one end and a graspable handle 24 on the opposing end, in which most or all of the elongate shaft 20 itself is configured to vibrate. In one embodiment, the elongate shaft 20 may be a thin, flexible, wire-like body that vibrates all along its length.

The motor 26 for powering the vibration of the probe tip 30 of the apparatus 10, in one embodiment, may include a commonly available vibrating motor, such as a piezoelectric element that converts current into an ultrasonic vibration, an unbalanced weight connected to the shaft 20 and rotating inside the probe tip 30 to create vibration, or any other mechanism sufficient to create the desired vibrations.

The frequency of vibration of the apparatus 10 of the present invention may be selected for the particular use and need not be in the ultrasonic range. Likewise, the amplitude of the vibrations created by the apparatus 10 may vary according to the intended use. In one embodiment, the apparatus 10 of the present invention may include a controller 29 coupled to the motor 26 whereby the user may vary the frequency and amplitude of the vibration. In this aspect, the apparatus 10 of the present invention may be used to deliver a vibration having any of a variety of frequencies and amplitudes, depending upon the intended use.

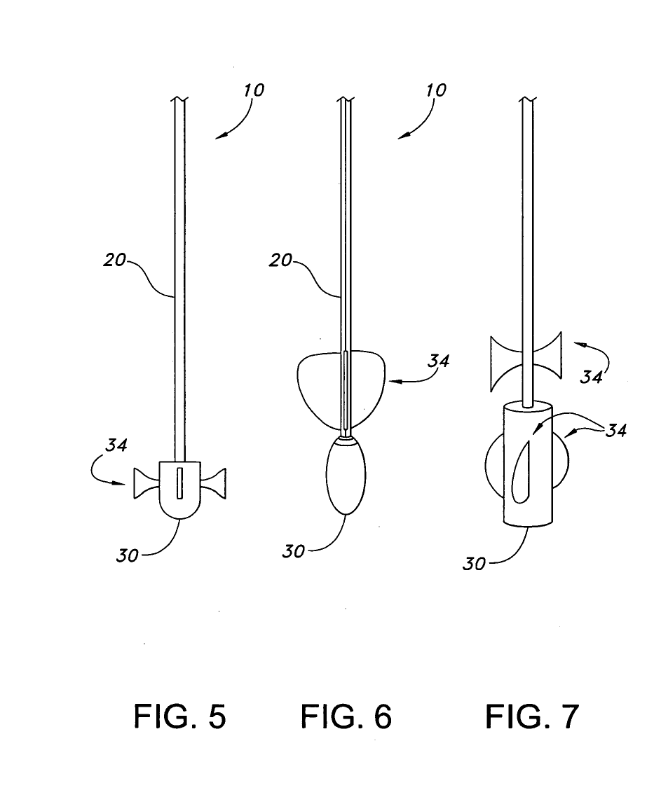

As shown in FIG. 4 and FIG. 5, the probe tip 30 in one embodiment may be shaped like a plug with a rounded distal end. In another embodiment, shown in FIG. 6, the probe tip 30 may be ellipsoidal or egg-shaped. As shown in FIG. 7, the probe tip 30 may be generally cylindrical. The probe tip 30 of the present invention may be any shape and size, depending upon the particular use and the size and shape of the cavity where the apparatus 10 will be used. For various uses, the probe tip shape may be ovoid, spherical, ellipsoidal, cylindrical, cubical, prismatic, spool-shaped, bell-shaped, a combination of these shapes, or an amorphous shape.

In one embodiment, the apparatus 10 of the present invention may include one or more probe tips 30 of different shapes and sizes, attachable to the apparatus 10 using a releasable attachment means, so the user may install the probe tip 30 desired for a particular application. The probe tips 30 may be disposable or designated for single-use only. In this aspect, a probe tip 30 may be selected to fit the size and shape of the femoral intramedullary canal 110 or other bony cavity where agitation is desired.

As shown in FIG. 4 and FIG. 5, the probe tip 30 in one embodiment may include one or more fins 34 extending outwardly into the fluid 40. The fins 34, in one embodiment, may be disposed upon the shaft 20, as shown in FIG. 6. As shown in FIG. 7, the fins 34 may be disposed on both the probe tip 30 and the shaft 20. The fins 34 may be distributed evenly about the shaft 20, as shown in FIG. 2. In another embodiment, the fins 34 may be distributed at irregular positions or intervals in order to produce eccentricity and a desired vibration.

In one aspect, the fins 34 may function to centralize the probe tip 30 or, in other words, keep the probe tip 30 generally centered within the canal 110, as shown in FIG. 4.

In one embodiment, the fins 34 may function to disrupt, coalesce into larger voids, and mobilize the entrapped air voids 44. Also, the fins 34 may be shaped to assist in keeping the probe tip 30 near the center of the canal 110, in order to deliver an evenly distributed vibration throughout the surgical fluid 40. The fins 34 may take any shape and size, depending upon the particular use and the size and shape of the cavity where the apparatus 10 will be used. In one embodiment, the apparatus 10 of the present invention, the one or more sets of probe tips 30 may include fins 34 of different shapes and sizes.

5. A Method of Agitating a Surgical Fluid

As shown in FIG. 1, the apparatus 10, in one embodiment, may be inserted through a reamed canal 208 into the interior of a bone 100. Depending upon the surgical procedure, access to the bone interior can be gained using any of a variety of locations for a reamed canal 208. In one embodiment, the reamed canal 208 may be placed through the side wall 120 of a bone (not shown), in order to avoid resecting a bone end 204, to avoid drilling through the epiphyseal plates, for example, or to otherwise reduce the trauma or forces exerted upon the bone 100 and the surrounding tissues.

As shown in a closer view in FIG. 4, the apparatus 10 of the present invention may include a cap 70 generally sealing the opening to the bone interior and a suction device 80 positioned through the cap 70. The cap 70, as shown, may include an opening sufficient to allow entry of the shaft 20, probe tip 30, and fins 34, while maintaining a sufficient seal. A relatively tight seal may be desired so the suction 80 will create a slight vacuum or pressure gradient.

As shown in a closer view in FIG. 4, a restrictor 320 may be inserted through the reamed canal 208 into the intramedullary canal 110 to serve as a stop or base for the column of surgical fluid 40 to be inserted. The fluid 40 may be referred to as a column because of its elongate shape and because the bone 100 may be oriented such that the surgical field is generally vertical. The restrictor 320 may be selected to fit the size and shape of the canal 110, and it may be made of any suitable material such as natural bone or a bioabsorbable gel.

In one embodiment, the restrictor 320 may include a lug 322 that may be configured to releasably receive a surgical instrument used for placing the restrictor 320 in a desired location. In one embodiment, the lug 322 may be sized and shaped to releasably receive the probe tip 30 on the end of the shaft 20 such that the apparatus 10 may be used to insert and install the restrictor 320 at the desired location. In this aspect, the insertion of the probe apparatus 10 may be used to accomplish the secondary function of installing the restrictor 320. Once in place, the restrictor 320 may be released from the probe tip 30.

Next, the method of the present invention may include cleansing the interior walls of the intramedullary canal 110 using, for example, high-pressure pulse lavage. The canal 110 may be packed with a dry sponge. In general, a clean and dry interior wall may improve the bonding of surgical fluid 40 and the bone wall 120.

Many surgical fluids 40 require the mixing of two or more components during the surgical procedure. The mixing step may be accomplished in an open container or within a fluid insertion device such as a cement gun or syringe.

Next, the method of the present invention may include the selection and assembly of a probe tip 30 and fins 34 to fit the size and shape of the intramedullary canal 110.

As shown in FIG. 4, the apparatus 10 may be inserted through either reamed canal 208 into a mass or column of surgical fluid 40. The column of surgical fluid 40 may entrap one or more air voids 44, as shown. The fluid 40 may be contained at the base by a restrictor 320 and on the sides by the bone wall 120. The method of agitating the surgical fluid 40 in vivo provides a benefit in that any air voids 44 introduced into the canal 110 by other procedures (such as injecting the fluid 40 or placing a prosthesis 350) may be removed before the fluid 40 hardens or cures.

In one aspect of the method, the apparatus 10 may be placed directly against the restrictor 320 or the bone wall 120 in order to cause the entire surgical field to vibrate. This technique may produce vibrations in the bone 100 and surrounding structures that will propagate inwardly from the bone walls 120 as well as outwardly from the probe tip 30, thus creating an improved environment for laminar fluid flow and increased interdigitation. Because the intramedullary canal 110 may be generally cone-shaped, this technique may be referred to creating a “sonic horn” inside the bone 100.

In one embodiment, the method of the present invention may include the steps of inserting a vibrating apparatus 10 into the surgical fluid 40, and vibrating the column of surgical fluid 40 in order to disrupt and mobilize the entrapped air voids 44 and drive them toward and through a surface of the fluid 40. As shown in FIG. 4, the apparatus 10 may include a cap 70 and a suction device 80 in order to create a partial vacuum or a decreasing pressure gradient across the column of surgical fluid 40. In this aspect, the step of driving the air voids 44 upward is accomplished in part because the vibrations of the apparatus 10 may cause the voids 44 to coalesce together and rise toward the surface, along the decreasing pressure gradient, against gravity, toward the vacuum created by the suction device 80. The buoyancy of the air voids 44 when exposed to the vibrational forces may be sufficient to overcome the viscosity of the surgical fluid 40, such that the air voids 44 may be moved through the fluid. In this way, the air voids 44 may be driven toward the surface of the fluid 40, where the air can escape the fluid 40.

In this aspect of the method, the apparatus 10 may be manipulated in the most effective motions, perhaps beginning near the restrictor 320 near the base of the column of surgical fluid 40 and moving generally upward toward the surface. In use, the patterns of motion and manipulation will vary depending upon the cavity or canal being filled, the size and shape of the fluid 40, the viscosity of the fluid 40 during the agitation, and other factors which necessarily vary depending upon the circumstances. The user may stop the probe tip 30 from vibrating before removing it from the fluid 40.

In another aspect of the present invention, the method in one embodiment may include the steps of inserting a vibrating apparatus 10 into the surgical fluid 40, and vibrating the column of surgical fluid 40 in order to drive the fluid 40 toward the endosteum 114 and into the plurality of bone pores 130 in the inner bone wall 120, as shown in FIG. 2. Although each individual pore 130 may be small, the cumulative effect of the increased interdigitation of the fluid 40 with multiple pores 130 results in an improved bond along the fluid-bone interface 50, resulting in more efficient load transfer and torsional strength. FIG. 2 is a cross-sectional illustration of the apparatus 10 in one embodiment, positioned within an intramedullary canal 110. In this aspect, the method of the present invention promotes and produces interdigitation between the surgical fluid 40 and the bone wall 120. The area where interdigitation occurs may be referred to as the bone-fluid interface 50. Although a single section of bone-fluid interface 50 is labeled in FIG. 2, the bone-fluid interface 50 exists around the entire inner surface of the bone wall 120. The use of a vibrating apparatus 10 according to the method of the present invention produces more interdigitation between the fluid 40 and the bone wall 120 than would otherwise be possible because the vibrational energy drives the fluid 40 into and among the nearby bone pores 130, where the fluid 40 may penetrate the pores 130, solidify, and form an interlocking relationship between the fluid 40 and the bone wall 120.

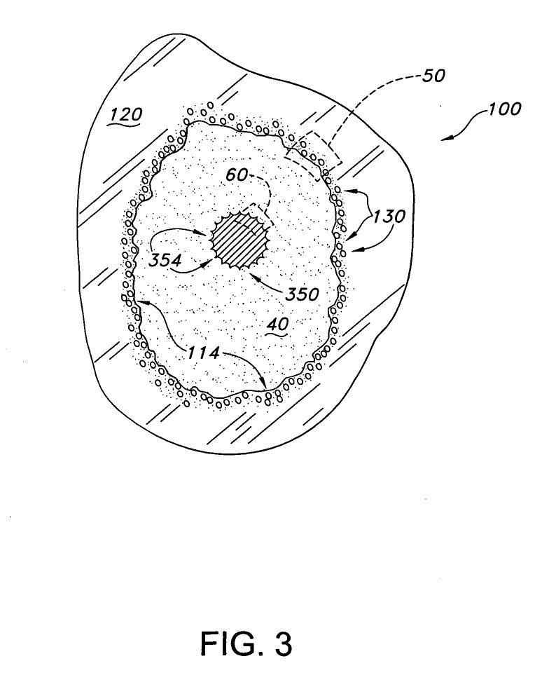

In another aspect of the present invention, the method in one embodiment may include the steps of inserting a vibrating apparatus 10 into the surgical fluid 40, and vibrating the column of surgical fluid 40 in order to drive the fluid 40 toward the surface of a prosthesis 350 and into a plurality of ridges 354 on the outer surface of the prosthesis 350, as shown in FIG. 3. FIG. 3 is a cross-sectional illustration of a prosthesis 350 positioned within an intramedullary canal 110. In this aspect, the method of the present invention promotes and produces interdigitation between the surgical fluid 40 and the prosthesis 350. The area where interdigitation occurs may be referred to as the fluid-prosthesis interface 60. Although a single section of fluid-prosthesis interface 60 is labeled in FIG. 3, the fluid-prosthesis interface 60 exists around the entire outer surface of the prosthesis 350. The use of a vibrating apparatus 10 according to the method of the present invention produces more interdigitation between the fluid 40 and the prosthesis 350 than would otherwise be possible because the vibrational energy drives the fluid 40 into and among the ridges 354 on the prosthesis 350, where the fluid 40 may more fully penetrate the ridges 354, solidify, and form an interlocking relationship between the fluid 40 and the prosthesis 350.

In another aspect, the apparatus 10 of the present invention may be used to improve the bond between the surgical fluid 40 and a prosthesis 350 having a smooth or polished surface. In this aspect, the method of the present invention removes air voids and promotes a smooth and uniform layer between the surgical fluid 40 and the prosthesis 350. In one embodiment, the method of the present invention may include heating the fluid 40 and the prosthesis 350 to a similar temperature. The fluid-prosthesis interface 60 in this aspect may be improved without the presence of pores or ridges in the prosthesis.

6. Hip Replacement Example

Implantation of a femoral prosthesis is one surgical procedure where the apparatus and method of the present invention may be applied. A femoral prosthesis 350 is illustrated in FIG. 1. In practicing the conventional cementing technique, the bone 100 may be prepared for the femoral prosthesis 350 in the standard fashion. Once the surgeon has determined the design and position of the prosthesis 350, the canal 110 may be prepared for cementation. A cement restrictor 320 of appropriate size and shape (shown in FIG. 4) may be placed about one centimeter distal to the measured length of the prosthesis 350. Care is taken not to oversize the restrictor 320 or to place it with excessive force. The canal 110 may then be cleansed with pulsatile lavage and packed with a dry sponge. During the time the surgical fluid 40 or cement is being mixed, the ambient temperature and humidity for the operating room may be noted, in order to estimate the proper curing time. Centrifugation or vacuum mixing may be performed on the cement mixture in order to reduce the porosity; in other words, to remove some of the entrapped air voids. At approximately four minutes after the initiation of mixing, depending upon the particular surgical fluid 40 being used, the fluid 40 or cement may be injected retrograde with a cement gun, allowing the pressure within the canal 110 to push out the nozzle of the cement gun. Using the pressure to push out the cement gun may help avoid the introduction of additional air into the fluid 40 from removing the nozzle too quickly. The column of surgical fluid 40 or cement may be proximally pressurized to improve the biomechanical characteristics of the cement mantle.

According to the method of the present invention, in one embodiment, the agitation of a viscous column of surgical fluid 40 may be used to increase the laminar flow characteristics of the fluid, thereby dispersing the entrapped air voids and promoting better interdigitation of the fluid 40 into nearby gaps and pores 130 in the bone wall, especially in trabecular and metaphyseal bone. In one aspect, as shown in FIG. 4, the improved interdigitation increases the surface area of the fluid-bone interface 50. Agitation of the surgical fluid 40 also improves the biomechanical characteristics of the fluid-prosthesis interface 60, thereby promoting more efficient transfer of forces and stresses from the prosthesis 350, through the fluid 40, to the bone 100.

In use, the method of the present invention, in one embodiment, eliminates the need for several steps currently used during fourth-generation cementing technique described above. In one aspect, pressurization of the column of surgical fluid 40 may be no longer required. Vibrating the column of surgical fluid 40 in vivo may provide more extrusion of the fluid 40 into surrounding bone pores 130 and prosthesis ridges 354 than would be provided by pressurization. The elimination of pressurization may result in a marked decrease in the incidence of fat emboli syndrome and may prevent the extrusion of the liquid monomer through the capillary membrane and into the circulation.

In another aspect, advance preparation of the surgical fluid 40 before insertion, by techniques such as vacuum mixing and centrifugation, may be eliminated when using the method of the present invention. In fact, the fluid 40 may be mixed directly in the barrel of the cement gun and immediately introduced into the bone cavity or canal 110 without waiting for curing. In this aspect, the method of the present invention reduces surgery duration, thereby reducing the time the patient may be under anesthesia and reducing the time the staff may be exposed to the sometimes noxious fumes from mixing a surgical fluid 40 in an open container.

7. Fracture Fixation Example

Fracture fixation and surgical repair of bone defects is another surgical procedure where the apparatus and method of the present invention may be applied. An intramedullary nail prosthesis 356 is illustrated in FIG. 1. In one current technique, an intramedullary nail 356 may be placed along the length of a fractured bone 100 to maintain alignment. The nail 356 may be secured by screws and will act as a load-bearing device until the fracture or other bone insult 200 is healed.

In one embodiment, the method and apparatus 10 of the present invention may be used to optimize the performance of a column of surgical fluid 40 in place of or in addition to the intramedullary nailing and related methods of fracture fixation. In this aspect, a column of surgical fluid 40 treated by the method and apparatus 10 of the present invention may have significantly fewer air voids 44 and increased interdigitation at the bone-fluid interface 50 such that a nail or other prosthesis 350 is not required for adequate fracture fixation. In another aspect, the treated column of surgical fluid 40 may be used together with a prosthesis 350. By and through the method and apparatus 10 of the present invention, the column of surgical fluid itself may improve its capacity to resist forces in compression and extension, torsion, bending, and shear. In this aspect, an optimal column of hardened surgical fluid 40 treated by the method and apparatus 10 of the present invention may provide an alternative to the internal nailing and external casting of bone fractures.

In any procedure where a surgical fluid 40 is used, such as to fill voids or bone defects with liquid bone material or grafts, the benefits of removing air voids 44 and increasing interdigitation at the bone-fluid interface 50 are many and may be achieved by agitation of the fluid 40 in vivo using the method and apparatus 10 of the present invention. The resulting, hardened column of surgical fluid 40, together with the adjacent bone wall 120 interconnected by and through the improved interdigitation, provides a support having improved biomechanical properties, such as better durability, increased strength, and structural integrity.

Fracture fixation using only a column of surgical fluid 40 is another surgical procedure where the apparatus and method of the present invention may be applied. In such a procedure, an expandable bone stent prosthesis surrounded by a flexible sheath may be placed at a fracture site within the intramedullary canal 110 and filled with a surgical fluid 40. These components may be introduced into the intramedullary canal 110 through an incision and a simple breach in the bone, without the need for example to resect the femoral head. Once in place, the cement or other surgical fluid 40 hardens and provides fixation and stabilization of the fracture site. The surgical fluid 40 may be contained at least partially by the sheath in order to prevent the fluid 40 from seeping into the fracture site. The vibrating apparatus 10 of the present invention may used to agitate the surgical fluid 40 in order to remove entrapped air voids and to improve interdigitation between the fluid 40 and the bone stent prosthesis.

8. Conclusion

The described embodiments of the invention are intended to be merely exemplary. Numerous variations and modifications will be apparent to those skilled in the art. All such variations and modifications are intended to fall within the scope of the present invention as defined in the appended list of exemplary inventive concepts.

What has been described above includes several examples. It is, of course, not possible to describe every conceivable combination of components or methodologies for purposes of describing the systems, methods, and apparatuses used for orthopaedic procedures. However, one of ordinary skill in the art may recognize that further combinations and permutations are possible. Accordingly, this application is intended to embrace alterations, modifications, and variations that fall within the scope of the appended list of exemplary inventive concepts. Furthermore, the preceding description is not meant to limit the scope of the invention. Rather, the scope of the invention is to be determined only by the appended list of exemplary inventive concepts and their equivalents.

While the systems, methods, and apparatuses herein have been illustrated by describing examples, and while the examples have been described in considerable detail, it is not the intention of the applicants to restrict or in any way limit the scope of the appended list of exemplary inventive concepts to such detail. Additional advantages and modifications will be readily apparent to those skilled in the art. Therefore, the invention, in its broader aspects, is not limited to the specific details, the representative systems and methods, or illustrative examples shown and described. Accordingly, departures may be made from such details without departing from the spirit or scope of the applicant's general inventive concepts.

1. A method of improving the structural integrity of a column of surgical fluid adjacent a bone wall having a plurality of open pores, the method comprising:
inserting a probe into said column of surgical fluid; and
vibrating said probe within said column of surgical fluid in order to drive entrapped air toward and through a surface of said fluid and to drive said surgical fluid into one or more of said plurality of open pores.

2. The method of claim 1, further comprising:
inserting a restrictor to form a base for said column of surgical fluid;
capping said column of surgical fluid near said surface; and
drawing a partial vacuum above said column of surgical fluid in order to help draw said entrapped air toward and through said surface.

3. The method of claim 2, further comprising:
touching said probe to said restrictor in order to induce vibrations within said restrictor and within said bone wall.

4. The method of claim 1, further comprising:
touching said probe to a bone wall in order to induce vibrations within said bone wall.

5. The method of claim 1, further comprising:
manipulating said probe from near the base of said column of surgical fluid toward said surface.

6. The method of claim 1, further comprising:
manipulating said probe near said bone wall in order to improve the depth of interdigitation between said fluid and said plurality of open pores.

7. The method of claim 1, further comprising:
cleansing said bone wall; and then
drying said bone wall before injecting said column of surgical fluid.

8. The method of claim 1, further comprising:
varying the frequency of vibration of said probe.

9. The method of claim 1, further comprising:
varying the amplitude of vibration of said probe.

10. A method of removing entrapped air from a column of surgical fluid, comprising:
inserting a probe into said column of surgical fluid; and
vibrating said probe within said column of surgical fluid in order to drive entrapped air toward and through a surface of said fluid.

11. The method of claim 10, further comprising:
inserting a restrictor to form a base for said column of surgical fluid;
capping said column of surgical fluid near said surface; and
drawing a partial vacuum above said column of surgical fluid in order to help draw said entrapped air toward and through said surface.

12. The method of claim 10, further comprising:
manipulating said probe from near the base of said column of surgical fluid toward said surface.

13. The method of claim 10, further comprising:
varying the frequency of vibration of said probe.

14. The method of claim 10, further comprising:
varying the amplitude of vibration of said probe.

15. A method of provoking interdigitation between a column of surgical fluid and a bone wall having a plurality of open pores, the method comprising:
inserting a probe into said column of surgical fluid; and
vibrating said probe within said column of surgical fluid in order to drive said surgical fluid into one or more of said plurality of open pores.

16. The method of claim 15, further comprising:
inserting a restrictor to form a base for said column of surgical fluid;
capping said column of surgical fluid near said surface; and
drawing a partial vacuum above said column of surgical fluid in order to help draw said entrapped air toward and through said surface.

17. The method of claim 16, further comprising:
touching said probe to said restrictor in order to induce vibrations within said restrictor and within said bone wall.

18. The method of claim 15, further comprising:
touching said probe to said bone wall in order to induce vibrations within said bone wall.

19. The method of claim 15, further comprising:
cleansing said bone wall; and then drying said bone wall before injecting said column of surgical fluid.

20. The method of claim 15, further comprising:
manipulating said probe near said bone wall in order to improve the depth of interdigitation between said fluid and said plurality of open pores.

21. The method of claim 15, further comprising:
varying the frequency of vibration of said probe.

22. The method of claim 15, further comprising:
varying the amplitude of vibration of said probe.

23. A vibrating probe apparatus for agitating a surgical fluid, comprising:
an elongate shaft having a proximal end and an opposing distal end, said proximal end being graspable for supporting and maneuvering the apparatus;
a probe tip disposed upon said shaft near said distal end; and
a motor for producing a vibration within said probe tip.

24. The apparatus of claim 23, wherein said motor produces a vibration along said elongate shaft.

25. The apparatus of claim 23, further comprising:
one or more fins disposed about said probe tip and extending into said fluid.

26. The apparatus of claim 23, further comprising:
one or more fins disposed about said elongate shaft and extending into said fluid.

27. The apparatus of claim 23, further comprising:
a graspable handle disposed upon said shaft near said proximal end for supporting and maneuvering the apparatus.

28. The apparatus of claim 23, further comprising:
a cable for coupling said motor to said apparatus.

29. The apparatus of claim 23, further comprising:
a controller coupled to said motor and configured to vary the frequency of said vibration.

30. The apparatus of claim 23, further comprising:
a controller coupled to said motor and configured to vary the amplitude of said vibration.

31. A system for bonding a column of surgical fluid to a bone wall, the system comprising:
a fluid restrictor positioned to form a base for said column of surgical fluid;
a plurality of open pores along said bone wall; and
a zone of interdigitation between said surgical fluid and said plurality of open pores, said zone promoted by the temporary insertion of a vibrating probe configured to drive said surgical fluid into one or more of said plurality of open pores,
said vibrating probe powered by a motor and comprising an elongate shaft having a graspable proximal end and an opposing distal end, a probe tip disposed upon said shaft near said distal end.

32. The system of claim 31, further comprising:
a partial vacuum zone adjacent an outer surface of said column of surgical fluid for drawing one or more air voids within said surgical fluid toward said outer surface,
said partial vacuum zone created by a suction device disposed through a cap at least partially sealing said column of surgical fluid near said outer surface.

33. The system of claim 32, wherein said vibrating probe mobilizes said one or more air voids toward said outer surface.

34. The system of claim 31, further comprising:
a lug disposed upon said fluid restrictor and configured to be releasably attached to said distal end of said elongate shaft.

35. A system for consolidating a prosthesis, a column of surgical fluid, and a porous bone wall into an integrated structure, the system comprising:
a fluid restrictor positioned to form a base for said column of surgical fluid;
a plurality of open pores along said bone wall;
a first zone of interdigitation between said surgical fluid and said plurality of open pores, said first zone promoted by the temporary insertion of a vibrating probe configured to drive said surgical fluid into one or more of said plurality of open pores; and
a second zone of interdigitation between the outer surface of said prosthesis and said surgical fluid, said second zone promoted by the temporary insertion of a vibrating probe configured to drive said surgical fluid into said outer surface;
said vibrating probe powered by a motor and comprising an elongate shaft having a graspable proximal end and an opposing distal end, a probe tip disposed upon said shaft near said distal end.

36. The system of claim 35, further comprising:
a partial vacuum zone adjacent an outer surface of said column of surgical fluid for drawing one or more air voids within said surgical fluid toward said outer surface,
said partial vacuum zone created by a suction device disposed through a cap at least partially sealing said column of surgical fluid near said outer surface.

37. The system of claim 36, wherein said vibrating probe mobilizes said one or more air voids toward said outer surface.

38. The system of claim 35, further comprising:
a lug disposed upon said fluid restrictor and configured to be releasably attached to said distal end of said elongate shaft.
